(19) United States
(12) Patent Application Publication
(10) Pub. No.: US 2007/0289596 A1
(43) Pub. Date: Dec. 20, 2007

(54) Endotracheal cuff and technique for using the same

(76) Inventors: Shannon E. Campbell, Oakland, CA (US); Joel C. Colburn, Walnut Creek, CA (US)

Correspondence Address: Nellcor Puritan Bennett LLC;c/o Fletcher Yoder PC, P.O. BOX 692289, HOUSTON, TX, 77269-2289, US

(21) Appl. No.: 11/453,161

(22) Filed: Jun. 14, 2006

(51) Int. Cl. A62B 9/06; A61M 16/00

(52) U.S. Cl. 128/207.15; 128/207.14

(57) ABSTRACT

An inflatable balloon cuff may be adapted to seal a patient's trachea via a mucoadhesive coating when associated with an endotracheal tube. The mucoadhesive seal may enhance a cuffs mechanical pressure seal, or may be used to seal the trachea at very low cuff inflation pressures. Also provided are inflatable balloon cuffs that include agents that may promote tracheal tissue growth and/or regeneration.

### BACKGROUND OF THE INVENTION

1. Field of the Invention

The present invention relates to medical devices, and more particularly, to endotracheal devices, such as endotracheal tubes and cuffs.

2. Description of the Related Art

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

In the course of treating a patient, a tube or other medical device may be used to control the flow of air, food, fluids, or other substances into the patient. For example, medical devices, such as tracheal tubes, may be used to control the flow of one or more substances into or out of a patient. In many instances, it is desirable to provide a seal between the outside of the tube or device and the interior of the passage in which the tube or device is inserted. In this way, substances can only flow through the passage via the tube or other medical device, allowing a medical practitioner to maintain control over the type and amount of substances flowing into and out of the patient.

Tracheal tubes may be used to control the flow of air or other gases through a patient's trachea. Such tracheal tubes may include endotracheal (ET) tubes, or tracheostomy tubes. To seal these types of tracheal tubes, an inflatable cuff may be associated with these tubes. When inflated, the cuff generally expands into the surrounding trachea to seal the tracheal passage around the circumference of the tube. A high-quality seal against the tracheal passageway allows a patient ventilator to perform efficiently.

As many patients are intubated for several days, healthcare workers may need to balance achieving a high-quality tracheal seal with possible patient discomfort. For example, the pressure of an inflated cuff against the tracheal walls may result in some discomfort for a patient. While a cuff may be inflated at lower pressure to avoid this discomfort, this may lower the quality of the cuff's seal against the trachea. Low cuff inflation pressures may also be associated with allowing folds to form in the walls of the cuff that may serve as leak paths for air as well as microbe-laden secretions.

Additionally, the quality of a cuff's seal against the tracheal passageway may suffer over the duration of a patient's intubation time. For example, a seal may be compromised when a patient coughs, which may dislodge the cuff from a sealed position. Further, when the endotracheal tube is jostled during patient transport or medical procedures, the force of the movement may shift the position of the inflatable cuff within the trachea, allowing gaps to form between the cuff and the tracheal walls.

### SUMMARY

Certain aspects commensurate in scope with the originally claimed invention are set forth below. It should be understood that these aspects are presented merely to provide the reader with a brief summary of certain forms the invention might take and that these aspects are not intended to limit the scope of the invention. Indeed, the invention may encompass a variety of aspects that may not be set forth below.

There is provided a medical device that includes an inflatable balloon cuff; and a mucoadhesive disposed on a tissue-contacting surface of the balloon cuff.

There is also provided a method that includes securing an inflatable balloon cuff to a patient's trachea with a mucoadhesive, wherein the inflatable balloon cuff is adapted to substantially seal the trachea.

There is also provided a method of manufacturing a medical device that includes providing an inflatable balloon cuff; and providing a mucoadhesive disposed on a tissue-contacting surface of the balloon cuff.

There is also provided a medical device that includes an inflatable balloon cuff; and a cilia growth-promoting agent disposed on a tissue-contacting surface of the balloon cuff.

There is also provided a method of promoting cilia regeneration that includes inflating a balloon cuff in a patient's trachea, wherein a tissue-contacting surface of the balloon cuff includes a therapeutically effective amount of a cilia growth-promoting agent.

There is also provided a method of manufacturing a medical device that includes providing an inflatable balloon cuff; and providing a cilia growth-promoting agent disposed on a tissue-contacting surface of the balloon cuff.

### BRIEF DESCRIPTION OF THE DRAWINGS

Advantages of the invention may become apparent upon reading the following detailed description and upon reference to the drawings in which:

FIG. 1 illustrates an endotracheal tube with an inflatable balloon cuff including a mucoadhesive layer in accordance with aspects of the present technique;

FIG. 2 illustrates the inflatable balloon cuff of the present techniques inserted into a patient's trachea;

FIG. 3 illustrates a hypothetical mucoadhesive seal;

FIG. 4 illustrates an endotracheal tube with a lumen for delivery of a solvent solution to a mucoadhesively sealed cuff; and

FIG. 5 illustrates an inflatable balloon cuff with a mucoadhesive layer that includes a vitamin.

### DETAILED DESCRIPTION OF SPECIFIC EMBODIMENTS

One or more specific embodiments of the present invention will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

It is desirable to provide a medical balloon, such as an endotracheal cuff or other medical device, which may have an improved seal when inserted into a patient's trachea. In accordance with some aspects of the present technique, a medical balloon with a mucoadhesive surface coating is provided that is adapted to be used with an endotracheal tube or device. Such a device may reversibly anchor an inflatable balloon cuff to the mucosal surfaces of a trachea, reducing movement of the cuff caused by coughing or outside forces acting on the endotracheal tube. Further, a mucoadhesive anchor to the tracheal walls may reduce “blow-by” air leaks along the sides of the cuff walls as well as decreasing microbe-laden secretions leaking past the cuff and entering the lung. The strength of the mucoadhesive seal may allow lower cuff inflation pressures to be used, which may result in reduced mechanical pressure against the tracheal walls. Additionally, medical devices for use in the trachea are provided that include tissue stimulation factors that may stimulate cilia growth and/or regeneration.

The term mucoadhesive refers to a substance that sticks to or adheres to the mucous membrane by any number of mechanisms, for example, but not limited to the following: hydrogen-bonding, ionic interaction, hydrophobic interaction, van der Waals interaction, or combinations thereof. While not intending to be bound in any way by theory, mucoadhesion may involve penetration of the of top layer mucosal tissue by the mucoadhesive and/or mechanical interlocking between mucin and the mucoadhesive substance. Generally, at physiological pH, a mucin glycoprotein carries a net negative charge. The surface attraction forces between the mucin and a positively charged mucoadhesive agent in the mucoadhesive may be responsible for the contact of the two surfaces and the adhesive strength. It is noted that additional explanations for mucoadhesion may also exist.

Provided herein are inflatable balloon cuffs that are mucoadhesive. Such balloon cuffs may be used in conjunction with any suitable medical device. In certain embodiments, the cuffs, as provided herein, may be used in conjunction with a catheter, a stent, a feeding tube, an intravenous tube, an endotracheal tube, a tracheostomy tube, a circuit, an airway accessory, a connector, an adapter, a filter, a humidifier, a nebulizer, or a prosthetic.

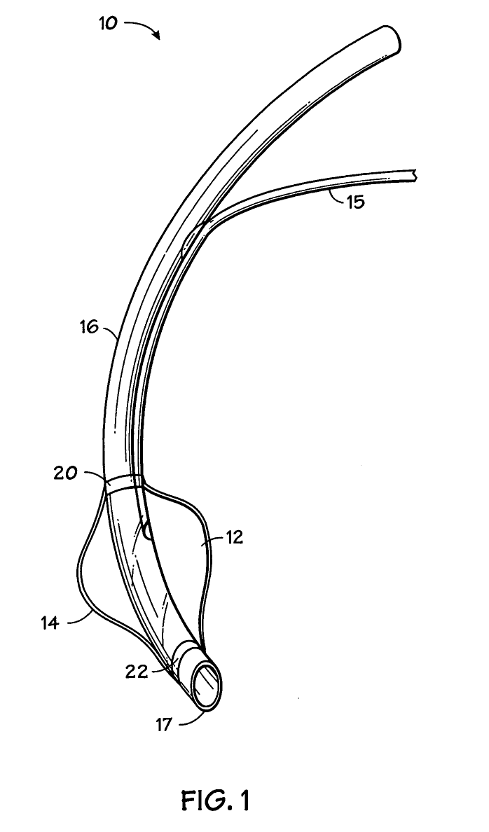

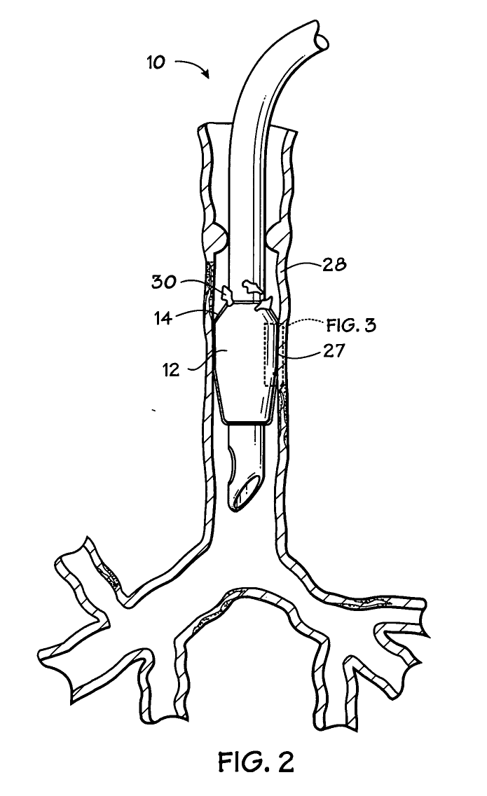

An example of an inflatable cuff used in conjunction with a medical device is an endotracheal tube 10, depicted in FIG. 1. The endotracheal tube 10 includes an inflatable cuff 12 that may be inflated to form a mucoadhesive seal against the trachea walls 28 (see FIG. 2). Disposed on the outer layer of the cuff 12 is a mucoadhesive layer 14. The cuff 12 is disposed on a conduit 16 that is suitably sized and shaped to be inserted into a patient and allow the passage of air through the conduit 16. Typically, the cuff is disposed, adhesively or otherwise, towards the distal end 17 of the conduit 16. The cuff 12 may inflated and deflated via a lumen 15 in communication with the cuff 12, typically through a hole or a notch in the conduit 16. The cuff 12 has a proximal opening 20 and a distal opening 22 formed in the cuff walls to accommodate the conduit 16.

The cuff 12 may be formed from materials having suitable mechanical properties (such as puncture resistance, pin hole resistance, tensile strength), chemical properties (such as forming a suitable bond to the tube 16), and biocompatibility. In one embodiment, the walls of the inflatable cuff 12 are made of polyurethane having suitable mechanical and chemical properties. An example of a suitable polyurethane is Dow Pellethane® 2363-90A. In another embodiment, the walls of the inflatable cuff 12 are made of a suitable polyvinyl chloride (PVC). Suitable materials may also include polyethylene teraphthalate (PETP), low-density polyethylene (LDPE), polypropylene, silicone, neoprene, or polyisoprene.

FIG. 2 shows the exemplary endotracheal tube 10 that has been inserted into a patient's trachea. The cuff 12 is inflated to form a mucoadhesive seal 27 against the tracheal walls 28 and may prevent secretions 30 or other detritus from passing through the trachea into the lungs. Typically, endotracheal cuffs are inflated within a patient's trachea such that the intra cuff pressure is approximately 20-30 cm H₂O. Endotracheal cuffs utilizing inflation pressures significantly greater than 25 cm H₂O, such as 100 cm H₂O, may be referred to as high-pressure cuffs, while cuffs that are designed to be inflated at pressures less than 25 cm H₂O may be considered low-pressure cuffs. It is envisioned that a mucoadhesive layer 14 may be included in both high-pressure cuffs and low-pressure cuffs. Generally, healthcare providers associate low-pressure cuffs with less discomfort for the patient. Mucoadhesive cuffs 12 as provided herein may adequately seal the trachea at very low pressures. As a mucoadhesive cuff 12 may seal the trachea through chemical interaction with the mucosal tissue, such a cuff 12 need not rely solely upon a mechanical seal that is achieved through the pressure of the cuff inflation against the tracheal walls 28. In certain embodiments, mucocadhesive cuffs 12 provide adequate tracheal sealing at very low intra cuff pressures. Thus, the cuff may be inflated at pressures low enough such that the mucoadhesive layer 14 on the cuff walls merely contacts the tracheal walls 28 in order to initiate the mucoadhesive seal 27. It is envisioned that a mucoadhesive cuff 12 may effectively seal a patient's trachea at intra cuff pressures less than 10 cm H₂O or less than 5 cm H₂O. In another embodiment, the cuff 12 may be briefly inflated at higher pressures to ensure a mucoadhesive seal 27 and then deflated to lower pressures after the mucoadhesive seal 27 has formed.

The strength of a mucoadhesive seal 27 may also be related to the total tracheal contact area of the cuff 12. Thus, it is envisioned that high-volume cuffs that maximize contact area with the tracheal walls may be particularly appropriate for such low-pressure sealing. High-volume cuffs may refer to cuffs that are designed to have diameters at least slightly larger than the tracheal diameter. Such a design may allow a tracheal cuff to be used with a wide variety of tracheal sizes. For example, a typical endotracheal cuff may be 1.5× the size of an average trachea when fully inflated. When such a high-volume cuff is inserted into a patient's trachea, the walls of the cuff may be unable to fully inflate in the tracheal passage. The cuff walls may fold to accommodate the excess volume in the cuff. If the cuff walls include a mucoadhesive coating, the folds may also provide additional surface area for tracheal contact. Thus, a mucoadhesive cuff may improve tracheal sealing of high-volume cuffs, as well as low-volume cuffs. It is envisioned that the mucoadhesive cuffs as provided herein may be 1.0×-2× the size of the tracheal diameter. It should be understood that the size of the average trachea may be an adult male trachea, an adult female trachea, a child trachea, and that these sizes may vary based on the height and weight of a patient.

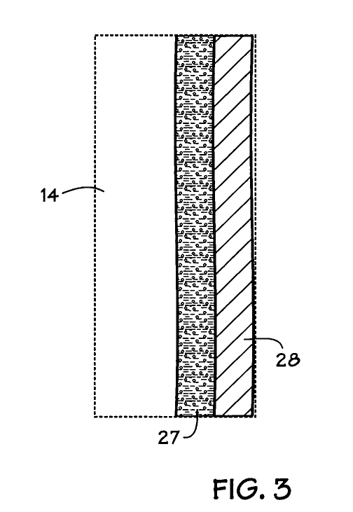

FIG. 3 illustrates an enlarged view of the mucoadhesive seal 27 against the tracheal walls 28. As depicted, the mucoadhesive layer 14 and the tracheal walls 28 form the mucoadhesive seal 27 at their interface. In certain embodiments, a patient's own mucosal fluid, which is formed mostly of water, will provide sufficient hydration to the mucoadhesive layer 14 to allow the seal to form. In other embodiments, a mucoadhesive agent may be mixed with water prior to the insertion of the cuff 12 into a patient's trachea or the water may be delivered to the cuff 12 after insertion via a lumen or other delivery mechanism. The adhesion strength of the mucoadhesive seal 27 may be characterized by its resistance to peeling, tensile strength, or adhesion time. In certain embodiments, it is envisioned that the mucoadhesive seal 27 may provide adequate anchoring of the cuff 12 for at least five days before degrading. In other embodiments, the relative strength of the mucoadhesive layer 14 may be characterized in vitro by measuring the strength of attachment to excised mammalian mucosal tissue using a surface tensiometer with a constant strain rate. In certain embodiments, the fracture energy of the mucoadhesive layer 14 from the tissue as measured by a surface tensiometer with a constant strain rate of 0.2 in s⁻¹ is at least 50 dyne cm⁻².

The mucoadhesive layer 14 may include a variety of mucoadhesive compositions and/or agents to secure a cuff to the mucosal tissue of the tracheal walls 28. Suitable mucoadhesives include, but are not limited to hydroxypropyl cellulose, hydroxypropyl methylcellulose, hydroxyethylcellulose, ethylcellulose, carboxymethylcellulose, dextran, cyclodextrins, guar gum, polyvinyl pyrrolidone, pectins, starches, gelatin, casein, acrylic acid polymers, polymers of acrylic acid esters, vinyl polymers, vinyl copolymers, polymers of vinyl alcohols, alkoxy polymers, polyethylene oxide polymers, polyethers, and any combination of the above.

In specific embodiments, the mucoadhesive may be a biocompatible polymer, for example polyacrylic acid, that is cross-linked with an acceptable agent to create an insoluble gel. The use of an insoluble gel may provide the advantage of adherence to the mucosal tissue for relatively long periods of time. For patients that experience longer intubation times, mucoadhesives such as cross-linked polyacrylic acid polymers, for example Noveon and Carbomer, may be appropriate for use for three to five days or longer. Noveon and Carbomer-based polymers are weak acids and contain many negatively-charged carboxyl-groups. The multiple negative charges on these polymers promote hydrogen-bonding between the polymers and the negatively mucin glycoproteins that mediate attachment of mucus to the epithelial lining. The mucoadhesive may also include chitosan, a deacetylated derivative of chitin, which is a natural biopolymer.

A mucoadhesive polymer may also include acrylic acid polymers (e.g. Carbopol® 940, also known as Carbomer® 940, Carbopol 934P and Carbopol® 980, products of BF Goodrich), methyl vinyl/maleic acid copolymers (e.g. Gantrez® S-97, a product of Internationl Specialty Products), polyvinyl pyrrolidone also known as povidone (e.g. Plasdone® K-90, a product of International Specialty Products). These polymers impart relatively high viscosity at relatively low concentrations. They may therefore be incorporated onto an inflatable cuff in amounts ranging from about 0.01% to about 10% by weight relative to the total composition. These viscosity modifying agents further act to improve the film adhesion of the composition to mucous membranes. Carbopol® 980, in certain embodiments, may be 2-3% by weight of the total composition.

The mucoadhesive layer 14 may be applied to the cuff 12 at the time of insertion into the trachea by a healthcare worker. In another embodiment, a mucoadhesive layer 14 may be applied to the cuff 12 after insertion via a lumen or other delivery mechanism. Alternatively, the mucoadhesive layer 14 may be incorporated directly onto the cuff 12. The mucoadhesive layer 14 may be applied to the surface of the cuff 12 by extruding, molding, dipping, spraying, washing, or painting, or any other suitable technique. The mucoadhesive may be formulated as a hydratable coating, a liquid, a dry powder, or a gel, depending on the desired viscosity. While certain mucoadhesives are available as gels or hydrogels, it is not necessary that the mucoadhesive layer 14 be as viscous as a gel, because a thin mucoadhesive layer 14 may provide certain manufacturing advantages. For example, a thin mucoadhesive layer 14 may be applied by dip-coating and subsequent curing. Further, depending on the strength of the particular mucoadhesive, thin mucoadhesive coatings may seal as well and for as long as gel coatings. If a liquid formulation or hydratable coating formulation is desired, a relatively low concentration (e.g. 0.1-1%) of the mucoadhesive/viscosity modifying agent may be used. If a gel formulation is desired, a higher concentration (e.g. 1.5-4%) of the suitable viscosity modifying/mucoadhesive agent may be incorporated into the polymethacrylate/solvent vehicle for gel formation. The mucoadhesive may further comprise excipients e.g. plasticizers, flavorings, sweeteners and/or colorants. Examples of plasticizers include triethyl citrate, polyethylene glycol and glycerin. Such plasticizers may be present in amounts generally ranging from about 1% to about 10% by weight relative to the total composition. For example, glycerine can be present in the amount of 1-5% by weight. Polyethylene glycol and triethyl citrate can be used in the amount of about 5% to about 12%, in certain embodiments.

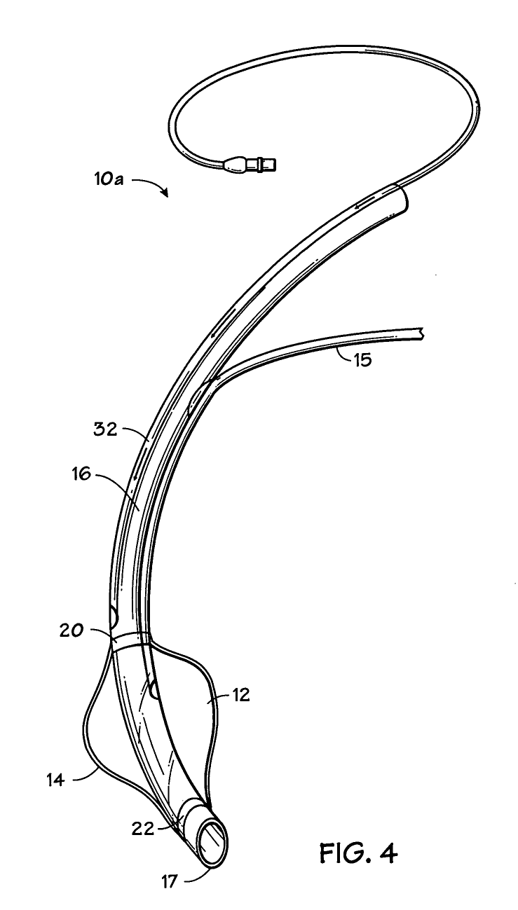

The mucoadhesive layer 14 may be adapted to degrade over time in the environment of the trachea. Alternatively, it is envisioned that a salt solution or any other suitable biocompatible mucoadhesive solvent may be flushed onto the cuff 12 prior to extubation to substantially weaken the mucoadhesive seal 27. FIG. 4 illustrates an alternative endotracheal tube 10a that includes a lumen 32 suitably sized and shaped to deliver a salt solution to a mucoadhesive seal 27. In a specific embodiment, the lumen 32 may be adapted to initially deliver a mucoadhesive to the cuff 12 and also subsequently deliver an appropriate solvent that is functional and biocompatible in order to dissolve the mucoadhesive. In certain embodiments (not shown), an endotracheal intubation kit may include a cuffed endotracheal tube 10a and a prefilled syringe (not shown) including an appropriate salt solution of a predetermined concentration.

It is also envisioned that an inflatable medical balloon, such as the cuffs 12, may include biologically active agents, for example agents adapted to promote cell growth or cilia growth. In such an embodiment, therapeutic agents such as growth factors may be included on the tissue-contacting surface of the cuffs as provided herein. Such agents may include therapeutically beneficial amounts of biologically active substances such as FGF (fibroblast growth factor), EGF (epidermal growth factor), PDGF (platelet-derived growth factor), IGF (insulin-like growth factor), TGF-β 1 through 3, cytokines, interferons, interleukins; hormones, insulin, growth hormone-releasing factor, calcitonin, and/or vitamins such as vitamin C, vitamin E, vitamin A or retinoic acid (e.g. trans-retinoic acid, 13-cis-retinoic acid, 9-cis-retinoic acid, other retinoids and mixtures thereof).

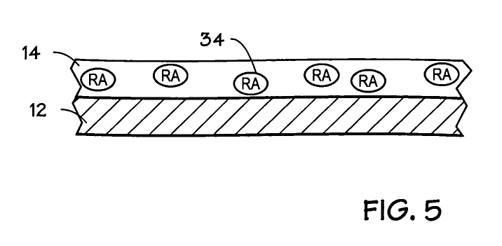

In a specific embodiment, a cuff 12 as provided herein may include a therapeutic quantity of a retinoic acid in order to promote cilia regeneration. As depicted in FIG. 5, a therapeutic agent such as a retinoic acid 34 may be incorporated into a mucoadhesive layer 14 disposed on a cuff 12. In certain embodiments, the mucoadhesive layer 14 includes at least 0.01% retinoic acid, or at least 0.025% retinoic acid. It should be understood that in other embodiments, the retinoic acid or other therapeutic agent may also be incorporated on the surface of a cuff 12 that does not include a mucoadhesive layer 14.

In other embodiments, it may be advantageous to provide a cuff 12 that includes a therapeutic agent or a combination of therapeutic agents with a wide variety of biological activities. For example, the agent may include anti-inflammatory, anticoagulant, antibiotic, antiallergic and antioxidant compounds. Examples of such anticoagulants include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIa platelet membrane receptor antagonist antibody, recombinant hirudin, and thrombin inhibitors such as Angiomax (Biogen, Inc., of Cambridge, Mass.). An example of an antiallergic agent is permirolast potassium. Other therapeutic substances or agents which may be appropriate include alpha-interferon, genetically engineered epithelial cells, rapamycin, dexamethasone, and functional analogs and structural derivatives thereof. The therapeutic agent may include peptides or proteins (such as enzymes, growth factors, hormones, and antibodies), small molecule compounds, nucleic acids, lipids, carbohydrates, steroids, glycoproteins, peptidomimetics, and/or oligodynamic metals.

The therapeutic agent may be applied to the surface of the cuff by techniques such as spraying, dipping, covalently bonding, extrusion blow-molding, or cross-linking the agent to the polymeric material of the cuff. For example, the cuff may be dip-coated by dipping the cuff in a solution containing the compound for a sufficient period of time (such as, for example, five minutes) and then drying the coated cuff, preferably by means of air drying for a sufficient period of time (such as, for example, 30 minutes). The agent may be chemically attached to the surface of the cuff through a two-step process involving cuff surface activation through energy activation (e.g. plasma, pulsed plasma, flow discharge reactive chemistry (FDRC), corona discharge) or chemical activation, and subsequently chemically coupling the agent to the activated surface. Such coupling of the agent to the cuff surface may be accomplished through carbodiimide chemistry, reductive amination, malemide-thiol reactions, etc. Further, the therapeutic agent may be compounded with a polymer composition and extruded or molded onto the surface of the cuff as an outer layer, or may be compounded into the cuff material itself.

In particular, the nature of the therapeutic agent may dictate its method of attachment to the cuff. For example, retinoic acids tend to be relatively hydrophobic, and thus generally insoluble in water. In order to incorporate a retinoic acid onto a relatively hydrophilic cuff surface, it may be advantageous to encapsulate the retinoic acid in amphipathic microspheres that shield the hydrophobicity of the retinoic acid from the hydrophilic polymer on the cuff walls. Such microspheres may enhance delivery of the retinoic acid to the mucosa. Proteins such as growth factors may also be encapsulated in microspheres to be applied to the surface of the cuff 12. Nanoparticles may also be used to encapsulate or assist in attachment of therapeutic agents to the cuff. Fullerenes, micelles or liposheres can all be functionalized to attach to specific surfaces and provide controlled-release of hydrophilic or hydrophobic molecules. Alternatively, dendromers can be assembled to contain specific binding sites or adhesion properties and allow for a high concentration of surface groups, which may include one or more therapeutic agents.

The therapeutically beneficial agent may be adapted to be released from the cuff 12 over time. For example, the therapeutic agents may be incorporated into a mucoadhesive layer 14 that is adapted to degrade over time, which may allow release of the therapeutic agent. In other embodiments, a therapeutic agent such as an antimicrobial agent may be adapted to be released over time via a water-soluble glass. In such an embodiment, a cuff 12 may include an antimicrobial metal such as silver in a phosphorus-based glass material that dissolves in water at a rate that may be a function of its particular formulation. In one embodiment, a calcium phosphorus-based glass may be part of a polymer layer that is made up of about 5-10% by weight, e.g. about 7.5% calcium phosphorus-based glass by weight. Such a phosphorus-based glass is available from Giltech Limited, 12 North Harbour Industrial Estate, Ayr, Scotland, Great Britain KA8 8BN.

The tracheal cuffs 12 of the present techniques may be incorporated into systems that facilitate positive pressure ventilation of a patient, such as a ventilator. Such systems may typically include connective tubing, a gas source, a monitor, and/or a controller. The controller may be a digital controller, a computer, an electromechanical programmable controller, or any other control system.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

What is claimed is:

1. A medical device comprising:
an inflatable balloon cuff; and
a mucoadhesive disposed on a tissue-contacting surface of the balloon cuff.

2. The medical device, as set forth in claim 1, wherein the mucoadhesive comprises an acrylic acid polymer, a methyl vinyl copolymer, or a polyvinyl pyrrolidone.

3. The medical device, as set forth in claim 1, wherein the mucoadhesive comprises a liquid, a dry powder, or a gel.

4. The medical device, as set forth in claim 1, wherein the balloon cuff comprises polyethylene teraphthalate (PETP), low-density polyethylene (LDPE), polyvinyl chloride (PVC), silicone, neoprene, polyisoprene, or polyurethane (PU).

5. The medical device, as set forth in claim 1, comprising an endotracheal tube associated with the balloon cuff, wherein the endotracheal tube passes through a proximal opening and a distal opening of the balloon cuff.

6. The medical device, as set forth in claim 5, comprising a ventilator to which the endotracheal tube is operatively connected.

7. The medical device, as set forth in claim 5, wherein the endotracheal tube comprises a lumen adapted to deliver a solvent to the inflatable cuff.

8. The medical device, as set forth in claim 1, wherein the mucoadhesive is adapted to degrade over time.

9. The medical device, as set forth in claim 1, wherein the mucoadhesive is adapted to dissolve upon contact with a solvent.

10. The medical device, as set forth in claim 1, wherein the mucoadhesive is adapted to have a tissue bond strength of at least 50 dyne cm⁻².

11. A method comprising:
securing an inflatable balloon cuff to a patient's trachea with a mucoadhesive, wherein the inflatable balloon cuff is adapted to substantially seal the trachea.

12. The method, as set forth in claim 11, comprising inflating the balloon cuff to an intra cuff pressure of less than 10 cm H₂0.

13. The method, as set forth in claim 11, comprising inflating the balloon cuff to an intra cuff pressure of less than 5 cm H₂0.

14. The method, as set forth in claim 11, wherein the balloon cuff is larger than 1.5 times the size of the patient's trachea.

15. The method, as set forth in claim 11, comprising detaching the balloon cuff by applying a solvent to the mucosal tissue.

16. The method, as set forth in claim 15, wherein the solvent is applied via a lumen associated with an endotracheal tube.

17. A method of manufacturing a medical device, comprising:
providing an inflatable balloon cuff; and
providing a mucoadhesive disposed on a tissue-contacting surface of the balloon cuff.

18. The method, as set forth in claim 17, wherein the mucoadhesive comprises an acrylic acid polymer, a methyl vinyl copolymer, or a polyvinyl pyrrolidone.

19. The method, as set forth in claim 17, wherein the mucoadhesive comprises a liquid, a dry powder, or a gel.

20. The method, as set forth in claim 17, wherein the balloon cuff comprises polyethylene teraphthalate (PETP), low-density polyethylene (LDPE), polyvinyl chloride (PVC), silicone, neoprene, polyisoprene, or polyurethane (PU).

21. The method, as set forth in claim 17, comprising providing an endotracheal tube associated with the balloon cuff, wherein the endotracheal tube passes through a proximal opening and a distal opening of the balloon cuff.

22. The method, as set forth in claim 21, wherein the endotracheal tube comprises a lumen adapted to deliver a solvent to the inflatable cuff.

23. The method, as set forth in claim 21, wherein the mucoadhesive is adapted to degrade over time.

24. The method, as set forth in claim 21, wherein the mucoadhesive is adapted to dissolve upon contact with a solvent.

25. The method, as set forth in claim 21, wherein the mucoadhesive is adapted to have a tissue bond strength of at least 50 dyne cm⁻².

26. A medical device comprising:
an inflatable balloon cuff; and
a cilia growth-promoting agent disposed on a tissue-contacting surface of the balloon cuff.

27. The medical device, as set forth in claim 26, wherein the balloon cuff comprises polyethylene teraphthalate (PETP), low-density polyethylene (LDPE), polyvinyl chloride (PVC), silicone, neoprene, polyisoprene, or polyurethane (PU).

28. The medical device, as set forth in claim 26, comprising an endotracheal tube associated with the balloon cuff, wherein the endotracheal tube passes through a proximal opening and a distal opening of the balloon cuff.

29. The medical device, as set forth in claim 28, wherein the endotracheal tube is operatively connected to a ventilator.

30. The medical device, as set forth in claim 26, wherein the cilia growth-promoting agent comprises a vitamin or a growth factor.

31. The medical device, as set forth in claim 26, wherein the cilia growth-promoting agent comprises a retinoic acid.

32. The medical device, as set forth in claim 26, wherein the cilia growth-promoting agent is covalently attached to the balloon cuff.

33. The medical device, as set forth in claim 26, wherein the cilia growth-promoting agent is embedded in a polymer layer disposed on the balloon cuff.

34. The medical device, as set forth in claim 26, wherein the balloon cuff comprises a mucoadhesive.

35. A method of promoting cilia regeneration comprising:
inflating a balloon cuff in a patient's trachea, wherein a tissue-contacting surface of the balloon cuff includes a therapeutically effective amount of a cilia growth-promoting agent.

36. The method, as set forth in claim 35, wherein the cilia growth-promoting agent comprises a vitamin or a growth factor.

37. The method, as set forth in claim 35, wherein the cilia growth-promoting agent comprises a retinoic acid.

38. The method, as set forth in claim 35, wherein the cilia growth-promoting agent is covalently attached to the balloon cuff.

39. The method, as set forth in claim 35, wherein the cilia growth-promoting agent is embedded in a polymer layer disposed on the balloon cuff.

40. The method, as set forth in claim 35, wherein the balloon cuff comprises a mucoadhesive.

41. A method of manufacturing a medical device, comprising:
providing an inflatable balloon cuff; and
providing a cilia growth-promoting agent disposed on a tissue-contacting surface of the balloon cuff.

42. The method, as set forth in claim 41, wherein the balloon cuff comprises polyethylene teraphthalate (PETP), low-density polyethylene (LDPE), polyvinyl chloride (PVC), silicone, neoprene, polyisoprene, or polyurethane (PU).

43. The method, as set forth in claim 41, comprising providing an endotracheal tube associated with the balloon cuff, wherein the endotracheal tube passes through a proximal opening and a distal opening of the balloon cuff.

44. The method, as set forth in claim 43, wherein the endotracheal tube is operatively connected to a ventilator.

45. The method, as set forth in claim 41, wherein the cilia growth-promoting agent comprises a vitamin or a growth factor.

46. The method, as set forth in claim 41, wherein the cilia growth-promoting agent comprises a retinoic acid.

47. The method, as set forth in claim 41, wherein the cilia growth-promoting agent is covalently attached to the balloon cuff.

48. The method, as set forth in claim 41, wherein the cilia growth-promoting agent is embedded in a polymer layer disposed on the balloon cuff.

49. The method, as set forth in claim 41, wherein the balloon cuff comprises a mucoadhesive.
